(19) United States
(12) Patent Application Publication
(10) Pub. No.: US 2024/0148919 A1
(43) Pub. Date: May 9, 2024

(54) NAVEL CLEANING DEVICE

(71) Applicants: Reed Y. Nelson, St. George, UT (US); Douglas Wirthlin, St. George, UT (US); Kevin D. Seely, Santa Clara, UT (US)

(72) Inventors: Reed Y. Nelson, St. George, UT (US); Douglas Wirthlin, St. George, UT (US); Kevin D. Seely, Santa Clara, UT (US)

(21) Appl. No.: 18/504,011

(22) Filed: Nov. 7, 2023

Related U.S. Application Data
(60) Provisional application No. 63/382,748, filed on Nov. 8, 2022.

(51) Int. Cl. A61L 2/00

(52) U.S. Cl. CPC A61L 2/0088

(57) ABSTRACT

A navel cleaning device features a housing, a capsule containing a cleaning agent, and a dispensing mechanism that can be actuated to release the cleaning agent. The device may include a base circumscribing an aperture of the housing, and may include an applicator adjacent to or within the aperture. Upon placing the navel cleaning device on the abdomen of a user, actuation of the dispensing mechanism releases cleaning agent into the applicator for delivery to the navel. After the cleaning process, a clinician or user may then remove any liquid or residue from the navel. The navel cleaning device provides a means to cleanse the navel prior to surgical procedures performed therethrough, leading to a reduction in the overall time and expense of the operation.

### CROSS-REFERENCE TO RELATED APPLICATIONS

This application claims the benefit of U.S. Provisional Patent Application No. 63/382,748 filed Nov. 8, 2022 and entitled “NAVEL CLEANING DEVICE,” which is incorporated herein by reference in its entirety.

### TECHNICAL FIELD

The present disclosure relates to hygienic means for cleaning the navel. More particularly, the present disclosure relates to a device for administering cleaning agents to the navel.

### BACKGROUND

The navel, referred to clinically as the umbilicus, and more commonly known as the belly button, is a scar in the middle of the abdomen that marks the point of former attachment to the umbilical cord during fetal development. While the navel is unique to each individual, the umbilical cord scar often manifests as either a depression or protrusion. The navel should be regularly cleaned with soap and warm water. In the absence of regular cleaning, the navel becomes a collection point for bacteria, dirt, sweat, soap, lotions, and lint. Left untreated, the navel may begin to develop infections and related smells, often paired with red, itchy, and scabby skin, especially in cases involving navel piercings. Some studies, for example, suggest that the average navel contains 67 different species of bacteria. As a result, there is a need for an efficient and hygienic means of cleaning the navel.

Many modern surgical procedures are performed through incisions in or around the patient's navel. Performing surgery through the navel can mean less scarring, reduced pain, lower blood loss, and a faster recovery than alternative means of laparoscopic surgery. As discussed above, however, many navels are unclean. Thus, prior to surgery, the navel passage must be thoroughly cleared and sanitized to mitigate the risk of introducing bacteria or other foreign elements within the surgical site. Currently, medical professionals administer several cotton swabs into the navel to remove any debris and sanitize the surgical site. The cost of a surgical unit on standby per minute of lost time cleaning out the navel amounts to hundreds of dollars. Further, cotton swabs may not thoroughly cleanse and disinfect the navel. Therefore, a need exists to cleanse the navel prior to surgical procedures performed therethrough, leading to a reduction in the overall time and expense of the operation. Likewise, a need exists for an easy and effective, routine hygienic cleaning method to prevent infections and growths within the navel. The invention disclosed herein solves these and other problems.

### BRIEF SUMMARY

In one aspect, a navel cleaning device, includes a housing, a capsule disposed within the housing and containing a cleaning agent, and a dispensing mechanism configured to be actuated to release the cleaning agent from the capsule.

The navel cleaning device may also include an aperture configured to provide an exit for the cleaning agent from the housing, and a base circumscribing the aperture.

The navel cleaning device may also include an adhesive layer configured to secure the housing to a periumbilical region of the subject.

The navel cleaning device may also include an applicator positioned within or adjacent to the aperture.

In some embodiments, the dispensing mechanism may include a first arm and a second arm, where the first arm and second arm are coupled to the housing on opposite sides of the capsule and are configured to be moved so as to cause rupture of the capsule.

In some embodiments, the dispensing mechanism may include a button configured to rupture the capsule upon application of a compressive force to the button. In some of such embodiments, the housing comprises the button.

In one aspect, a method of cleaning a navel of a subject includes obtaining a navel cleaning device that includes a housing, a capsule disposed within the housing and containing a cleaning agent, and a dispensing mechanism configured to be actuated to cause the capsule to release the cleaning agent. The method also includes securing the navel cleaning device to a periumbilical region of the subject so that the capsule is situated over the navel. The method also includes actuating the dispensing mechanism to release the cleaning agent onto the navel. In some embodiments, actuating the dispensing mechanism releases the cleaning agent into an applicator for delivery to the navel.

In some embodiments, where the dispensing mechanism includes a button configured to rupture the capsule upon application of a compressive force to the button, the actuating step can includes applying the compressive force to the button.

In some embodiments, where the dispensing mechanism includes a first arm and a second arm coupled to the housing on opposite sides of the capsule, the actuating step includes moving the first arm and second arm so as to cause rupture of the capsule.

Other technical features may be readily apparent to one skilled in the art from the following figures, descriptions, and claims.

### BRIEF DESCRIPTION OF THE DRAWINGS

The embodiments disclosed herein will become more fully apparent from the following description and appended claims, taken in conjunction with the accompanying drawings. These drawings depict only typical embodiments, which will be described with additional specificity and detail through use of the accompanying drawings in which:

FIG. 1 shows a top, rear perspective view of a navel cleaning device.

FIG. 2 shows a top, side perspective view of a navel cleaning device adhered to a patient.

FIG. 3 shows a bottom, side perspective view a navel cleaning device.

FIG. 4 shows a rear, side perspective view of a navel cleaning device having a sponge.

FIG. 5 shows a glass capsule containing a cleaning agent.

FIG. 6 shows a top, front, side perspective view of a cylindrical embodiment of a navel cleaning device.

FIG. 7 shows a bottom plan view of a cylindrical embodiment of a navel cleaning device.

FIG. 8 shows a top perspective view of a rectangular planar embodiment of a navel cleaning device.

FIG. 9 shows a bottom perspective view of a rectangular embodiment of a navel cleaning device.

FIG. 10 shows a side elevation view of a rectangular embodiment of a navel cleaning device.

FIG. 11 shows an exploded bottom perspective view of an embodiment of a navel cleaning device.

FIG. 12 shows a side view of an embodiment of a navel cleaning device.

### DETAILED DESCRIPTION

The following descriptions depict only example embodiments and are not to be considered limiting in scope. Any reference herein to “the invention” is not intended to restrict or limit the invention to exact features or steps of any one or more of the exemplary embodiments disclosed in the present specification. References to “one embodiment,” “an embodiment,” “some embodiments,” “various embodiments,” and the like, may indicate that the embodiment(s) so described may include a particular feature, structure, or characteristic, but not every embodiment necessarily includes the particular feature, structure, or characteristic. Further, repeated use of the phrase “in one embodiment,” or “in an embodiment,” do not necessarily refer to the same embodiment, although they may.

Reference to the drawings is done throughout the disclosure using various numbers. The numbers used are for the convenience of the drafter only and the absence of numbers in an apparent sequence should not be considered limiting and does not imply that additional parts of that particular embodiment exist. Numbering patterns from one embodiment to the other need not imply that each embodiment has similar parts, although it may.

Accordingly, the particular arrangements disclosed are meant to be illustrative only and not limiting as to the scope of the invention, which is to be given the full breadth of the appended claims and any and all equivalents thereof. Although specific terms are employed herein, they are used in a generic and descriptive sense only and not for purposes of limitation. Unless otherwise expressly defined herein, such terms are intended to be given their broad, ordinary, and customary meaning not inconsistent with that applicable in the relevant industry and without restriction to any specific embodiment hereinafter described. As used herein, the article “a” is intended to include one or more items. When used herein to join a list of items, the term “or” denotes at least one of the items, but does not exclude a plurality of items of the list. For exemplary methods or processes, the sequence and/or arrangement of steps described herein are illustrative and not restrictive.

It should be understood that the steps of any such processes or methods are not limited to being carried out in any particular sequence, arrangement, or with any particular graphics or interface. Indeed, the steps of the disclosed processes or methods generally may be carried out in various sequences and arrangements while still falling within the scope of the present invention.

The term “coupled” may mean that two or more elements are in direct physical contact. However, “coupled” may also mean that two or more elements are not in direct contact with each other, but yet still cooperate or interact with each other.

The terms “comprising,” “including,” “having,” and the like, as used with respect to embodiments, are synonymous, and are generally intended as “open” terms (e.g., the term “including” should be interpreted as “including, but not limited to,” the term “having” should be interpreted as “having at least,” the term “includes” should be interpreted as “includes, but is not limited to,” etc.).

As previously discussed, there is a need for a means to cleanse the navel prior to surgical procedures performed therethrough, leading to a reduction in the overall time and expense of the operation and ensuring thorough cleaning and sterilization. Likewise, a related need exists for an easy and effective, routine hygienic cleaning method at home to prevent infections and growths within the navel. A navel cleaning device according to the present disclosure may solve these and other problems.

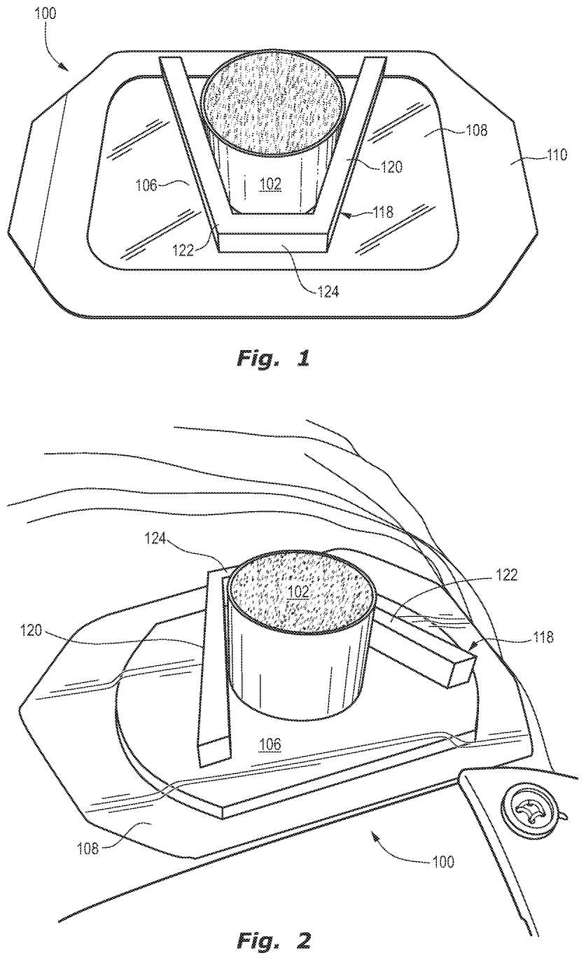

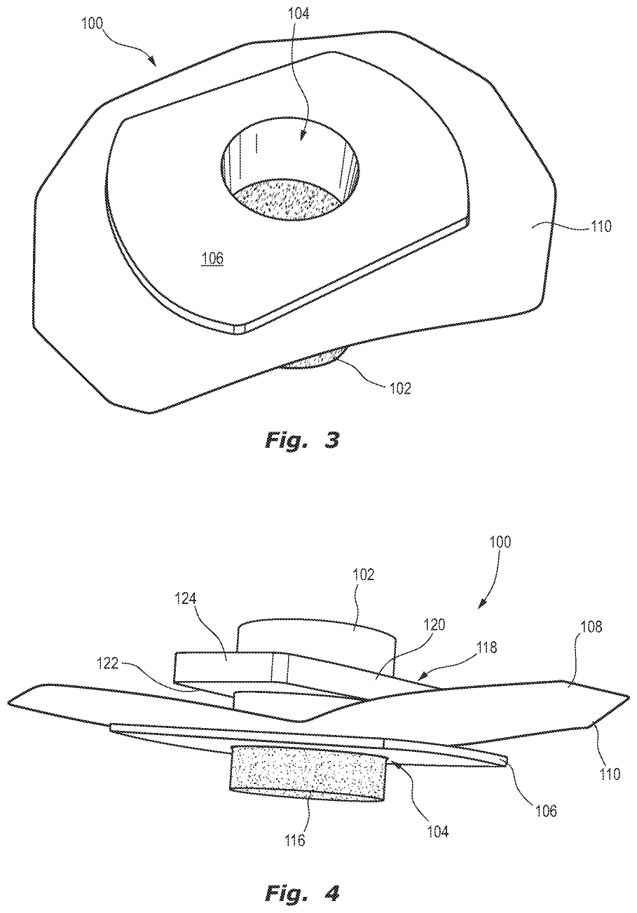

In some embodiments, as shown in FIG. 1 to FIG. 4, a navel cleaning device 100 can comprise a housing 102 having an aperture 104 and a base 106. As shown in FIG. 3, the housing 102 may comprise the aperture 104 circumscribed by the base 106. The base 106 may further comprise an adhesive layer 108 configured to secure the navel cleaning device 100 to the abdomen, particularly the periumbilical region, of a subject. The adhesive layer 108 can be coextensive with at least one surface of the base 106 and a release liner 110 coupled to the adhesive layer 108. In some embodiments, the adhesive layer 108 can include a pressure-sensitive adhesive such as, but not limited to, formulations based upon acrylates, natural rubbers, synthetic rubbers, vinyl acetates, or silicones. The adhesive layer 108 can also be flexible such that it can conform to the contours of the skin and form of the patient. In some embodiments, the adhesive layer 108 provides a waterproof, sterile barrier preventing external contaminants, including liquids, bacteria, bodily fluids, and viruses, into the navel. The adhesive layer 108 and release liner 110 may each circumscribe the exterior of the housing 102 or alternatively may circumscribe the aperture 104 of the housing 102. Both configurations of the adhesive layer 108 permit the filtration of a cleaning agent from within the aperture 104, directionally from the housing 102 to the navel. In some embodiments, the adhesive layer 108 may comprise a dressing with an exterior frame that flexes with the skin for greater patient comfort.

Devices according to the present disclosure can comprise a capsule containing a cleaning agent. The capsule can be configured to be sufficiently frangible to allow the capsule to be crushed, broken, torn, or punctured by a manual force applied to the device by a user. The capsule may comprise a frangible material such as glass, plastic, ceramic, metallic foil or other materials having a comparative impact strength. In some embodiments, as shown in FIG. 5, a capsule 112 may be an ampoule or similar sealed and sterilized vessel capable of housing a cleaning agent 114. In some embodiments, the capsule 112 may be a packet. In some embodiments, the capsule may comprise a membrane that can be punctured or ruptured to effect release of the cleaning agent. The cleaning agents can be liquid. Cleaning agents used with the devices described herein may comprise chlorhexidine, isopropyl alcohol, hydrogen peroxide, iodine, octinidine, triclosan, chloroxylenol, soap, saline (e.g., sterile saline), water (e.g., sterile water), other known antimicrobials and disinfectants, or mixtures thereof. Other cleaning agents can also be used.

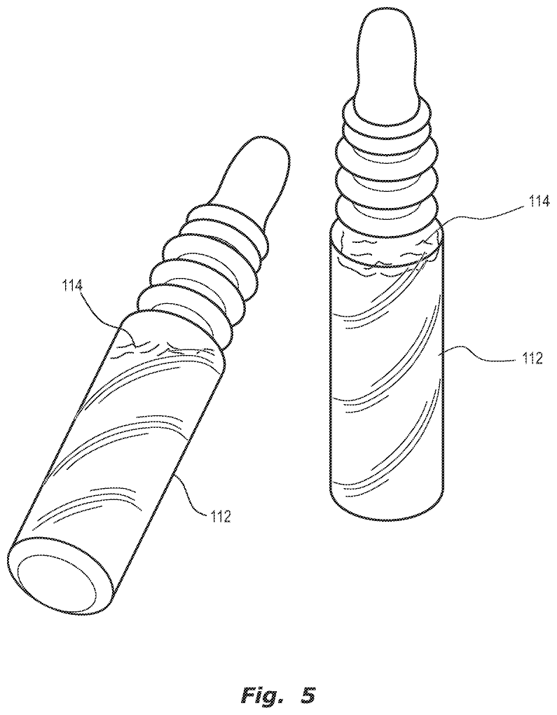

Referring again to the embodiment illustrated in FIG. 1 to FIG. 4, a capsule may be disposed within the housing 102 or otherwise coupled to an interior surface of the housing 102. In some embodiments, as shown in FIG. 4, an applicator 116, sponge, membrane, or other similar structure may be positioned adjacent to or within the aperture 104 to deliver the cleaning agent 114 to the navel. The applicator 116 can be configured to create a selective barrier preventing glass or other material from the capsule from leaving the housing 102 while still permitting the cleaning agent to filter across the applicator 116 and into the navel. The applicator 116 may comprise a sterile gauze pad or other fabric with an open weave structure tight enough to prevent glass solids from crossing from one side of the fabric to the other, yet be permeable to the cleaning agent. Other types of porous materials can also be used for the applicator 116. The applicator 116 can also be made of various materials, including cellulose or various polymers such as polyvinyl alcohol.

As further shown in FIG. 1 to FIG. 4, the applicator 116 can be disposed such that it extends below and away from the adhesive layer 108. When the navel cleaning device 100 is disposed on a patient, the applicator 116 can thus extend at least partially into the patient's navel area. The applicator 116 can also comprise a flexible material such that it may conform to the patient's navel during use, and it may be comfortable when actively used to scrub or wipe a patient's navel. The applicator 116 can also comprise an absorbent material such that it can at least temporarily absorb and retain the cleaning agent during use. The applicator 116 can deliver the cleaning agent to the patient's navel. For example, the absorbed cleaning agent can diffuse out of the applicator 116 (e.g., like a sponge). The applicator 116 can also release the cleaning agent at an increased rate when pressure is applied.

The navel cleaning device 100 can comprise a dispensing mechanism for effecting release of cleaning agent from the capsule. As shown in FIG. 1 and FIG. 2, the dispensing mechanism can comprise a bracket 118 coupled to the exterior surface of the housing 102. The bracket 118 may include a first arm 120 and a second arm 122 opposite the first arm 120. The first and second arms 120, 122 may be coupled to one another by a linking member 124. The first and second arms 120, 122 may be tangential to the housing 102 at an acute angle such that any exertion of force upon the first and second arms 120, 122 is leveraged against the exterior surface of the housing 102. The linking member 124 supports the first and second arms 120, 122 maintaining adherence to the exterior surface of the housing 102. The first and second arms 120, 122 of the bracket 118 are configured to permit a clinician or user to squeeze them together, thereby compressing the housing 102, rupturing the capsule within and releasing the cleaning agent into the navel.

In various embodiments, the housing may comprise a deformable material, particularly where actuation of the dispensing mechanism involves compression of the housing. Such materials include, without limitation, aluminum, tin, plastic, resin, silicone, rubber and the like. Without limiting the size and shape of the device, the housing 102 may be cylindrical or substantially cylindrical in shape, wherein the aperture 104 is circular. The housing 102 may optionally include a funnel that further directs the cleaning agent towards the navel.

In some embodiments, a method of using a navel cleaning device 100 comprises securing the housing 102 to the periumbilical region of the subject so that the capsule is situated over the navel. This can comprise detaching the release liner 110 from adhesive layer 108 of the navel cleaning device 100, adhering the adhesive layer 108 around the navel of a user, exerting force on an exterior surface of a housing 102 (such as via the bracket 118), releasing a cleaning agent 114 from a capsule housed within an aperture 104 of the housing 102 into the applicator 116 and to the navel, and then optionally removing any fluid and residue from the navel. A clinician may clean or scrub the abdominal region prior to adhering the adhesive layer 108 in order to improve adhesion to the skin. In some examples, the navel cleaning device may be utilized as soon as the patient is supine, allowing the cleaning agent to begin soaking and disinfecting the navel area. The navel cleaning device 100 can also be used passively in which it is applied to a patient and allowed to rest while cleaning agent 114 diffuses from the applicator 116 and to the navel. The navel cleaning device 100 can also be used actively in which a clinician may wipe or scrub the patient's navel, such as by grasping and moving an external surface of the housing 102 thereby moving the applicator 116 in and around the patient's navel area.

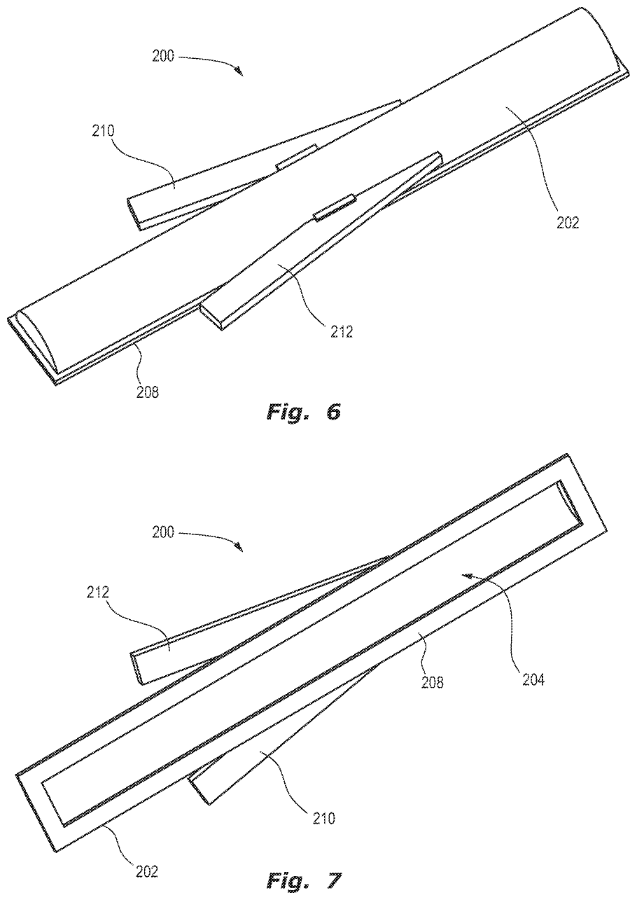

It will be appreciated that a navel cleaning device according to the present disclosure may comprise various shapes and configurations. For example, as shown in FIG. 6 to FIG. 7, a navel cleaning device 200 may comprise a housing 202 being semicylindrical in shape and having an aperture 204 extending along the longitudinal axis of the housing 202. The aperture 204 may be circumscribed by a base 208. In said embodiments, the housing 202 may comprise a first arm 210 and a second arm 212 configured to squeeze or compress the housing 202 when force is applied to the exterior of each arm 210, 212, thereby crushing a capsule or similar container within the housing 202, allowing the cleaning agent to exit the aperture 204 and contact the subject. As appreciated, an adhesive layer and release liner (not shown) such as described above may likewise be disposed on the base 208 of the cleaning device 200. The base 208 can also comprise an applicator (not shown), as described above with respect to FIG. 1 to FIG. 4.

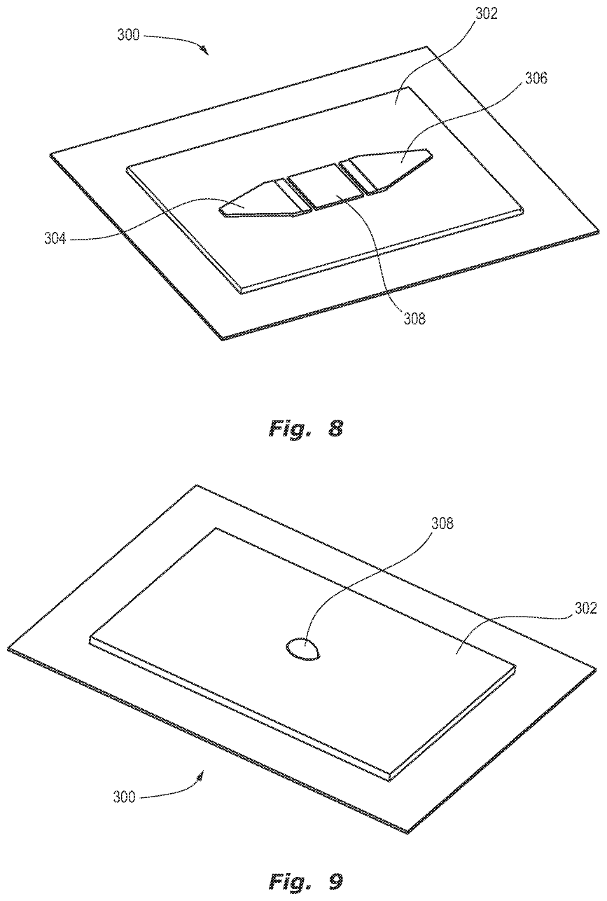

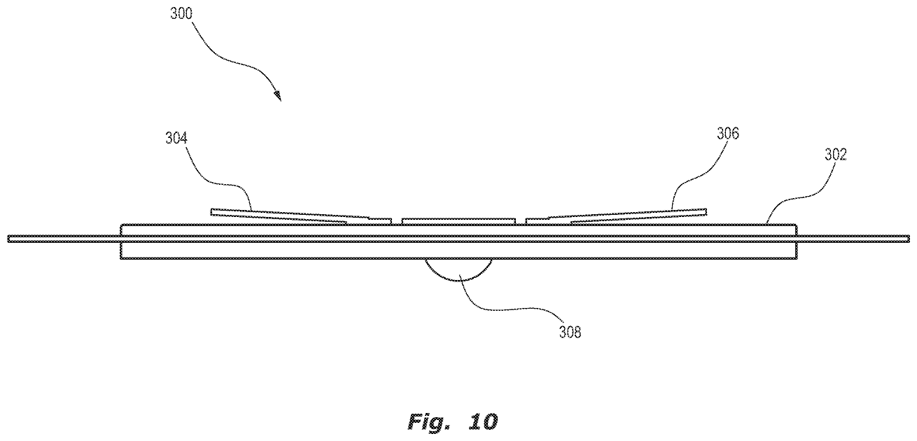

As shown in FIG. 8 through FIG. 10, a navel cleaning device 300 can comprise a housing 302, a first arm 304, a second arm 306, and a capsule 308. The first and second arms 304, 306 may be actuated by a user to crush or otherwise release a cleaning agent from within the capsule 308. The arms 304, 306 may be coupled to the capsule 308 so that movement of the arms causes rupture of the capsule 308. For example, the arms 304, 306 may be coupled to opposite points of the capsule 308, such that pulling apart, squeezing together, or rotating the arms 304, 306 deforms the capsule 308, causing it to rupture. Cleaning agent may then be released from the ruptured capsule 308 to contact the subject. As shown, the housing 302 may be rectangular and substantially planar and configured to hold the capsule 308. However, the housing 302 may comprise other shapes or configurations without departing herefrom. As will be appreciated, an adhesive layer and release liner as described above may likewise be disposed on a surface of the housing 302 to secure the navel cleaning device 300 to the abdomen of a subject. The navel cleaning device 300 can further comprise an applicator as described above with respect to FIG. 1 to FIG. 4.

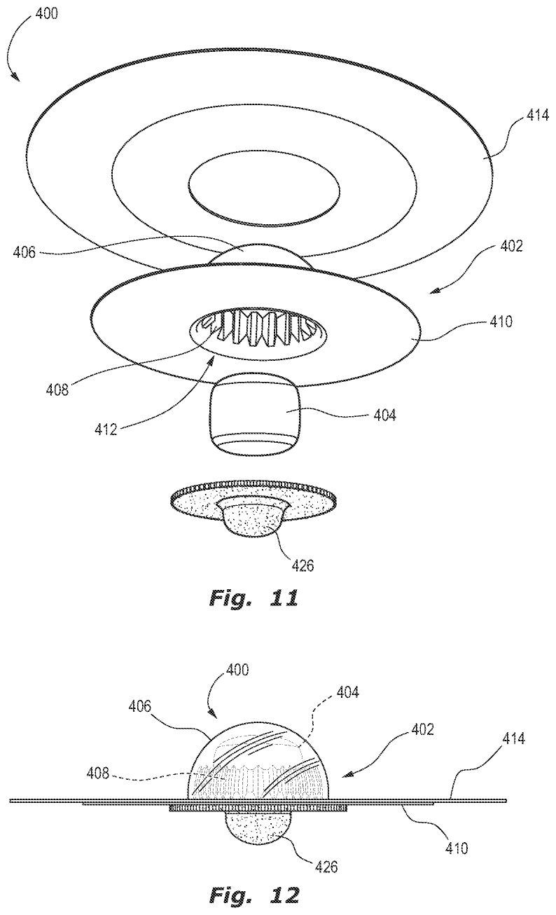

As shown in FIG. 11 and FIG. 12, a navel cleaning device 400 can comprise a housing 402 configured to enclose a capsule 404 containing a cleaning agent. A dispensing mechanism may be incorporated into the housing 402. For example, the housing 402 can include a button 406 that can be actuated to rupture the capsule 404 and effect release of the cleaning agent. The button 406 may be a flexible or pliable region of the housing 402, where actuation can be accomplished by manually applying a compressive force to the button 406. For example, the button 406 can be configured so that when pressed or squeezed by a user, an inner surface of the button 406 crushes the capsule 404. If desired, the inner surface of the button 406 may include rupture features 408 configured to facilitate rupture of the capsule 404. Such rupture features 408 may include ridges or other protuberances, such as spikes, knobs or bumps, configured to convey a force applied to the button 406 to the capsule 404. A base 410 may circumscribe an aperture 412 situated to provide an exit from the housing 402 for released cleaning agent. An applicator 426 may be positioned adjacent to or within the aperture 412 to deliver the cleaning agent. The applicator 426 may also provide a selective barrier permitting the cleaning agent to filter across the applicator 426 and to the navel while retaining the capsule within the housing (similar to the applicator described with respect to FIG. 1 to FIG. 4).

As shown, FIG. 11 is an exploded view of the navel cleaning device 400. FIG. 12 is a side view of the navel cleaning device 400, depicting the capsule 404 and rupture features 408 in phantom within the housing 402 and button 406. As can be appreciated, the rupture features 408 can be disposed within the housing 402 and button 406 in various ways, such as circumferentially (as shown), or partially on the top, sides, or combinations thereof.

The navel cleaning device 400 can further comprise an adhesive layer 414 such as described above to secure the device to the abdomen of a subject, particularly the periumbilical region. The adhesive layer 414 can be configured to provide a waterproof, sterile barrier preventing external contaminants from entering the navel when the navel cleaning device 400 is in place. The adhesive layer 414 can be coextensive with at least one surface of the base 410. In some embodiments, the device may include a release liner that is coextensive with an adhesive present on the adhesive layer 414.

Therefore, it will be appreciated that a navel cleaning device as described herein provides a means to cleanse the navel prior to surgical procedures performed therethrough, leading to a reduction in the overall time and expense of the operation. For example, hospitals and surgical centers may utilize the navel cleaning device 100 during surgical preparation or administration of anesthesia thereby saving valuable operating time at the beginning of the actual procedure. While a surgical application had been primarily discussed, the navel cleaning device also provides a self-applied, routine hygienic cleaning method at home to prevent infections and growths within the navel.

It will be appreciated that systems and methods according to certain embodiments of the present disclosure may include, incorporate, or otherwise comprise properties or features (e.g., components, members, elements, parts, and/or portions) described in other embodiments. Accordingly, the various features of certain embodiments can be compatible with, combined with, included in, and/or incorporated into other embodiments of the present disclosure. Thus, disclosure of certain features relative to a specific embodiment of the present disclosure should not be construed as limiting application or inclusion of said features to the specific embodiment unless so stated. Rather, it will be appreciated that other embodiments can also include said features, members, elements, parts, and/or portions without necessarily departing from the scope of the present disclosure.

Moreover, unless a feature is described as requiring another feature in combination therewith, any feature herein may be combined with any other feature of a same or different embodiment disclosed herein. Furthermore, various well-known aspects of illustrative systems, methods, apparatus, and the like are not described herein in particular detail in order to avoid obscuring aspects of the example embodiments. Such aspects are, however, also contemplated herein.

Exemplary embodiments are described above. No element, act, or instruction used in this description should be construed as important, necessary, critical, or essential unless explicitly described as such. Although only a few of the exemplary embodiments have been described in detail herein, those skilled in the art will readily appreciate that many modifications are possible in these exemplary embodiments without materially departing from the novel teachings and advantages herein. Accordingly, all such modifications are intended to be included within the scope of this invention.

What is claimed is:

1. A navel cleaning device, comprising:
a housing;
a capsule disposed within the housing and containing a cleaning agent; and
a dispensing mechanism configured to be actuated to release the cleaning agent from the capsule.

2. The navel cleaning device of claim 1, wherein the housing further comprises:
an aperture configured to provide an exit for the cleaning agent from the housing; and
a base circumscribing the aperture.

3. The navel cleaning device of claim 2, further comprising an applicator.

4. The navel cleaning device of claim 1, further comprising an adhesive layer configured to secure the housing to a periumbilical region of the subject.

5. The navel cleaning device of claim 4, wherein the adhesive layer includes a pressure-sensitive adhesive formulation.

6. The navel cleaning device of claim 4, further comprising a release liner coupled to the adhesive layer.

7. The navel cleaning device of claim 1, wherein the dispensing mechanism comprises a first arm and a second arm, wherein the first arm and second arm are coupled to the housing on opposite sides of the capsule and are configured to be moved so as to cause rupture of the capsule.

8. The navel cleaning device of claim 1, wherein the housing has a shape selected from cylindrical, semicylindrical, or planar.

9. The navel cleaning device of claim 1, wherein the dispensing mechanism comprises a button configured to rupture the capsule upon application of a compressive force to the button.

10. The navel cleaning device of claim 9, wherein the button includes an inner surface comprising one or more protuberances configured to receive the compressive force applied to the button and convey said compressive force to the capsule.

11. The navel cleaning device of claim 3, wherein the applicator comprises an absorbent, porous material.

12. The navel cleaning device of claim 3, wherein the applicator is flexible and is configured to extend at least partially into a navel area of a patient during use.

13. The navel cleaning device of claim 12, wherein the applicator comprises a sponge.

14. The navel cleaning device of claim 1, wherein the capsule comprises a frangible material.

15. The navel cleaning device of claim 14, wherein the frangible material is selected from glass, plastic, ceramic, and metallic foil.

16. The navel cleaning device of claim 1, wherein the cleaning agent comprises one or more of chlorhexidine, isopropyl alcohol, hydrogen peroxide, iodine, octinidine, triclosan, chloroxylenol, soap, saline, or sterile water.

17. The navel cleaning device of claim 1, wherein the housing comprises a deformable material selected from aluminum, tin, plastic, resin, silicone, and rubber.

18. A navel cleaning device, comprising:
a housing;
an adhesive layer configured to adhere the navel cleaning device to a patient;
a capsule disposed within the housing and containing a cleaning agent; and
an applicator configured to deliver the cleaning agent to a patient's navel,
wherein the housing comprises a deformable material, and wherein application of a compressive force to the housing can rupture the capsule thereby releasing the cleaning agent from the capsule to the applicator.

19. A method of cleaning a navel of a subject, comprising:
obtaining a navel cleaning device comprising:
a housing;
a capsule disposed within the housing and containing a cleaning agent; and
a dispensing mechanism configured to be actuated to cause the capsule to release the cleaning agent;
securing the navel cleaning device to a periumbilical region of the subject so that the capsule is situated over the navel;
actuating the dispensing mechanism to release the cleaning agent onto the navel.

20. The method of claim 19, wherein the dispensing mechanism comprises a button configured to rupture the capsule upon application of a compressive force to the button, and wherein the actuating step comprises applying the compressive force to the button.
